(19) United States
(12) Patent Application Publication
(10) Pub. No.: US 2022/0031203 A1
(43) Pub. Date: Feb. 3, 2022

(54) Systems and Methods for Detecting Cerebrospinal Fluid

(71) Applicant: Board of Regents, The University of Texas System, Austin, TX (US)

(72) Inventor: Mohamed Ibrahim, Friendswood, TX (US)

(21) Appl. No.: 17/389,794

(22) Filed: Jul. 30, 2021

Related U.S. Application Data
(60) Provisional application No. 63/058,655, filed on Jul. 30, 2020.

(51) Int. Cl. A61B 5/145; A61B 5/1468; A61B 5/00; A61M 25/00; A61M 39/02; A61M 39/24

(52) U.S. Cl. CPC A61B 5/14507; A61B 5/1468; A61B 5/4839; A61B 5/6852; A61M 25/00; A61M 2039/0241; A61M 39/24; A61M 2025/0007; A61M 2039/0205; A61M 2202/0464; A61M 2205/584; A61M 39/0208

(57) ABSTRACT

In one embodiment, a cerebrospinal fluid detection system includes a detection device including a detection compartment containing one or more indicators configured to indicate whether or not fluid received within the compartment contains cerebrospinal fluid and a port configured to pass fluid aspirated from a patient into the compartment.

### CROSS-REFERENCE TO RELATED APPLICATION

This application claims priority to co-pending U.S. Provisional Application Ser. No. 63/058,655, filed Jul. 30, 2020, which is hereby incorporated by reference herein in its entirety.

### BACKGROUND

In certain circumstances, it is necessary to access the epidural space within the spine. For example, medication is often delivered to the epidural space when providing continuous analgesia to a patient. Generally speaking, there are two primary methods used to confirm placement of a needle within the epidural space during needle advancement into the spine: the hanging drop method and the loss-of-resistance method. In practice, most anesthesiologists use the loss-of-resistance method.

In the loss-of-resistance technique, a needle is first inserted into the interspinous ligament or ligamentum flavum and a syringe containing an air bubble in saline is attached to the hub. After compression of the air bubble by increasing pressure using the plunger of the syringe that supports the needle, the needle is carefully advanced until a loss of resistance from the plunger is observed. When that occurs, it is concluded that the tip of the needle is within the epidural space. At that point, a catheter can be inserted into the space.

After accessing what is believed to be the epidural space, a test dose is typically administered to the patient as further confirmation that the tip of the catheter is within the space. Before the test dose is administered, however, the anesthesiologist normally aspirates the site in which the catheter tip is located to see if blood or cerebrospinal fluid (CSF) is collected. If so, this also provides confirmation that the epidural space has been accessed.

When aspiration is performed and fluid is collected, it is not always apparent whether the fluid is saline (used for loss-of-resistance detection), the anesthetic, or CSF. In such cases, it is not clear whether the catheter is in fact within the epidural space. In this scenario, anesthesiologists treat the catheter as if it is within subarachnoid space and administer the test dose beginning in very small quantities. For example, the anesthesiologist may first administer only one tenth of the typical dose and then increase the dose in further one-tenth increments until the target dose is reached. The reason for this is that even small doses of anesthesia can cause significant adverse effects to the patient, such as numbness of legs and loss of motor control, when it is administered in the subarachnoid space.

In view of the difficulty in knowing whether aspirated fluid is CSF or something else, it can be appreciated that it would be desirable to have means for detecting CSF.

### BRIEF DESCRIPTION OF THE DRAWINGS

The present disclosure may be better understood with reference to the following figures. Matching reference numerals designate corresponding parts throughout the figures, which are not necessarily drawn to scale.

FIG. 1A is a front view of a first embodiment of a system for detecting cerebrospinal fluid (CSF), including a detection device.

FIGS. 1B and 1C are first and second side views of the detection device shown in FIG. 1A.

FIG. 2A is a front view of a second embodiment of a system for detecting CSF, including a detection device.

FIGS. 2B and 2C are first and second side views of the detection device shown in FIG. 2A.

FIG. 3 is a front view of a third embodiment of a system for detecting CSF, including a detection device.

FIG. 4 is a side view of a fourth embodiment of a system for detecting CSF, including a detection device.

### DETAILED DESCRIPTION

As described above, it would be desirable to have means for detecting cerebrospinal fluid (CSF), for example, when aspirating the epidural space prior to administering anesthesia. Examples of such means are disclosed herein. As described below, a CSF detection method comprises analyzing the fluid that is collected during aspiration of what is believed to be the epidural space. In some embodiments, a CSF detection device is used that includes a test chamber in which the aspirated fluid is received that contains one or more indicators that provide an indication of when the fluid contains CSF. In some embodiments, the indicators comprise one or more chemical indicator elements that change color when the fluid comprises a particular pH and/or a particular substance commonly found in CSF.

In the following disclosure, various specific embodiments are described. It is to be understood that those embodiments are example implementations of the disclosed inventions and that alternative embodiments are possible. Such alternative embodiments include hybrid embodiments that include features from different disclosed embodiments. All such embodiments are intended to fall within the scope of this disclosure.

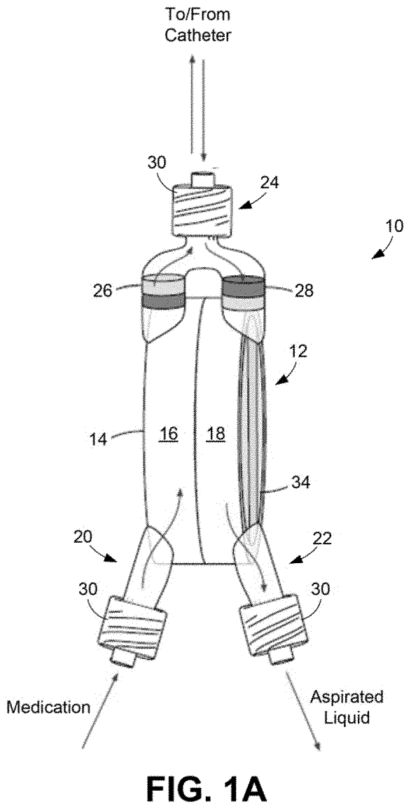

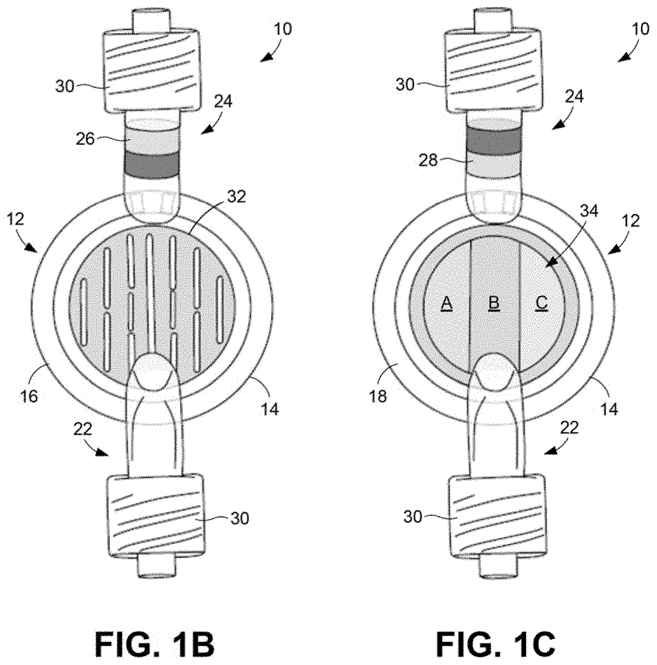

FIGS. 1A-1C illustrate an example system 10 for detecting CSF. Beginning with FIG. 1A, the system 10 includes a detection device 12 that can be placed in fluid communication with a catheter that extends to what is believed to be a patient's epidural space. As shown in that figure, the device 12 comprises a container 14, which, in this example, is configured as a short cylindrical container having a horizontally aligned central axis. The container 14 comprises two different compartments: a filter compartment 16 and a detection compartment 18. Provided within the filter compartment 16 is an air filter 32 that is used to filter out air bubbles within the anesthesia before it is delivered to the catheter. FIG. 1B is a first side view of the device 12 that illustrates the filter 32 within the filter compartment 16. By way of example, the filter 32 is a 0.2 μm medication filter. In fluid communication with the filter compartment 16 is an inlet 20, which can be used to deliver medication into the filter compartment.

With reference to FIG. 1C, the detection compartment 18 contains one or more indicators, in the form or one or more chemical indicator elements 34, that are used to detect the presence of CSF. In the illustrated embodiment, there are three separate chemical indicator elements 34, which are labeled A, B, and C. Notably, the presence of the substances can be inferred via the detection of the pH of the fluid and/or the detection of one or more substances normally contained in CSF, such as glucose and/or protein. In some embodiments, the chemical indicator elements 34 can comprise commercially available chemical indicator strips.

In regard to pH, a chemical indicator element 34 can change color to indicate the pH of the fluid. If the pH is within the pH range of CSF, i.e., 7.28-7.32, the presence of CSF is confirmed and access of the epidural space is presumed. [Are you certain that range of pH encompasses all CSF? I saw online numbers as high as 7.5.] SF normally contains approximately 50-80 mg/dl of glucose. In some embodiments, glucose detection can be based on the enzymatic glucose oxidase/peroxidase (GOD/POD) method. The reaction utilizes the enzyme glucose oxidase to catalyze the formation of gluconic acid and hydrogen peroxide from the oxidation of glucose. In turn, a second enzyme, peroxidase, catalyzes the reaction of hydrogen peroxide with the chromogen tetramethylbenzidine to form a green dye complex. A positive reaction is indicated by a color change from yellow to green. If this occurs, the presence of CSF is confirmed and access of the epidural space is presumed.

CSF normally contains 15 to 45 mg/dl of protein. In some embodiments, the detection of protein is based on the “protein error of pH indicators” (Sörensen, 1909). An example indicator element that can be used in this test is 3′,3″,5′,5″ tetrachlorophenol-3,4,5,6-tetrabromosulfophthalein. A positive reaction is indicated by a color change from yellow to light green/green. If this occurs, the presence of CSF is confirmed and access of the epidural space is presumed.

Referring back to FIG. 1A, in fluid communication with the detection compartment 18 is a drain 22, which can be used to drain aspirated fluid from the compartment.

With further reference to FIG. 1A, the container 14 also comprises a catheter port 24 that is in fluid communication with both the filter compartment 16 and the detection compartment 18. Separating the port 24 from the filter compartment 16 is a first one-way valve 26 that enables fluid to pass from that compartment to the catheter within the patient, but prevents fluid from passing back into the compartment. Separating the port 24 from the detection compartment 18 is a second one-way valve 28 that enables fluid to pass from the catheter to that compartment, but prevents fluid from the compartment to pass into the catheter. As is also shown in FIG. 1A, a connector 30 is provided at the free end of each of the inlet 20, the drain 22, and the catheter port 24. In some embodiments, each connector 30 comprises a standard Luer lock connector.

In operation, anesthesia and/or other medication can be injected into the filter compartment 16 via the inlet 20. This medication can then pass through the filter 32, through the first one-way valve 26, through the catheter port 24, and then into the catheter for delivery to the patient. When fluid is aspirated, a vacuum is applied to the drain 22 to draw fluid from the catheter through the catheter port 24, through the second one-way valve 28, and into the detection compartment 18 in which the fluid contacts the one or more chemical indicator elements 34. If CSF is within the fluid, the one or more chemical indicator elements 34 will change color and the location of the catheter within the epidural space is confirmed.

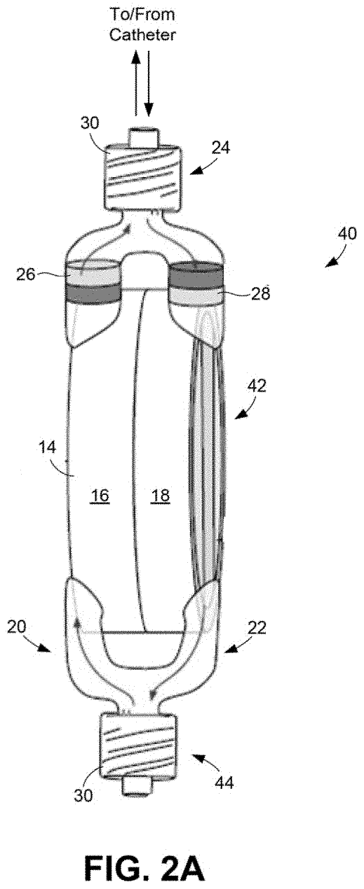

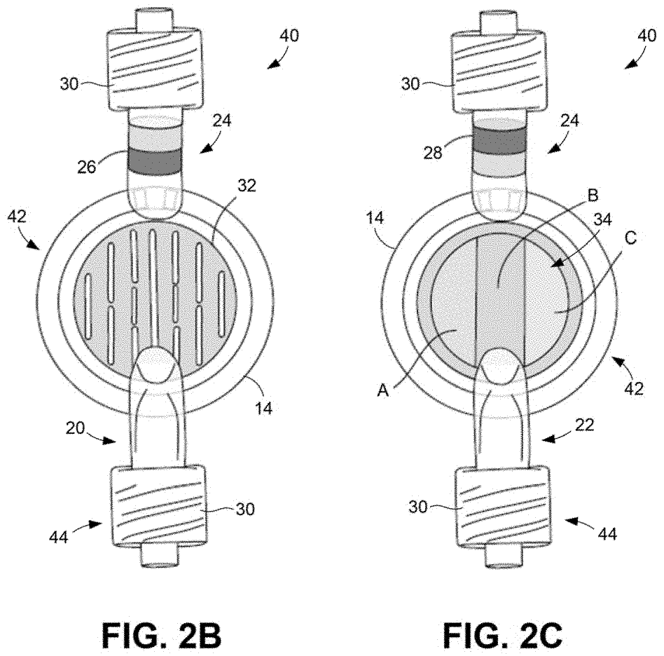

FIGS. 2A-2C illustrate a further example system 40 for detecting CSF. As with the system 10, the system 40 includes a detection device 42 that can be placed in fluid communication with a catheter that extends to what is believed to be a patient's epidural space. The detection device 42 is similar to the device 12 and, therefore, comprises several of the same components, which have the same reference numerals and which will not be discussed again. In this embodiment, however, the inlet 20 and the drain 22 combine with each other to form a single port 44 positioned at the bottom of the container 42 that can be used as both a port to deliver medication to the patient and as a drain to drain aspirated fluid collected from the patient. Other than this difference, the device 42 is substantially the same as the device 12 and operates in substantially the same way.

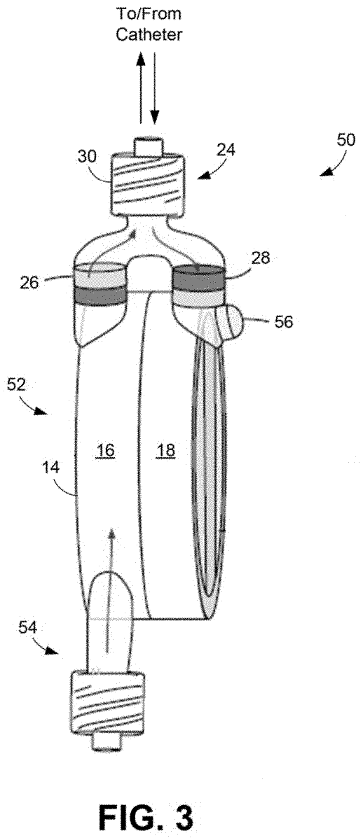

FIG. 3 illustrates another example system 50 for detecting CSF. As with the system 40, the system 50 includes a detection device 52 that can be placed in fluid communication with a catheter that extends to what is believed to be a patient's epidural space. The detection device 52 is similar to the device 42 and, therefore, comprises several of the same components, which have the same reference numerals and which will not be discussed again. In this embodiment, however, a single port 54 is connected to the filter compartment 16 (without being connected to the detection compartment 18). As is apparent from FIG. 3, a vacuum seal valve 56 is provided downstream of the second one-way valve 28 that leads to the detection compartment 18. The valve 56 contains a seal that ruptures when a threshold amount of pressure is applied to the seal. Once the seal is broken, a vacuum in the filter compartment 16 will aspirate back from the epidural catheter to enable drainage and detection of the presence or absence of CSF fluid.

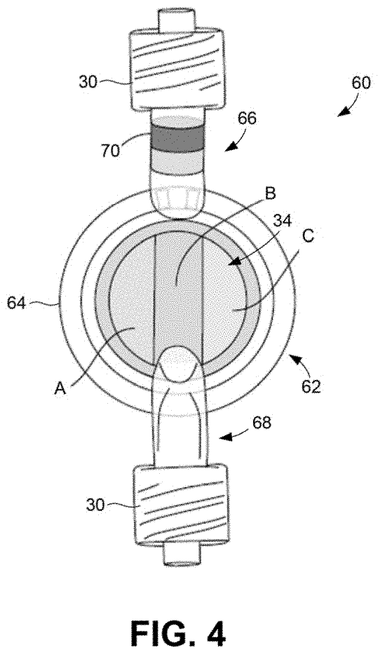

FIG. 4 illustrates yet another example system 60 for detecting CSF. The system 60 also includes a detection device 62 that, like the other detection devices described above, comprises a detection compartment 64 that contains one or more one or more chemical indicator elements 34. In this embodiment, however, the system 60 provides no filtration for the medication that is delivered to the patient and, therefore, comprises no filter compartment, filter, or inlet for the medication. Instead, the system 60 comprises a stand-alone detection device 62 having a dedicated port 66 through which aspirated fluid can enter the detection compartment 64, and a dedicated drain 68 through which the aspirated fluid can exit the detection compartment. As in the other embodiments, a one-way valve 70 is provided within the port 66 that enables fluid to pass from the catheter to that compartment, but prevents fluid from the compartment to pass into the catheter. The system 60 can be used in conjunction with an independent filtration device or an injection port.

Claimed are:

1. A cerebrospinal fluid detection system comprising:
a detection device including a detection compartment containing one or more indicators configured to indicate whether or not fluid received within the compartment contains cerebrospinal fluid and a port configured to pass fluid aspirated from a patient into the compartment.

2. The system of claim 1, wherein the one or more indicators comprise one or more chemical indicator elements configured to indicate when cerebrospinal fluid comes into contact with the chemical indicator elements.

3. The system of claim 2, wherein the one or more chemical indicator elements include a chemical indicator element configured to change color if the aspirated fluid from the patient has a pH within a pH range of cerebrospinal fluid.

4. The system of claim 2, wherein the one or more chemical indicator elements include a chemical indicator element configured to change color if the aspirated fluid from the patient contains glucose.

5. The system of claim 2, wherein the one or more chemical indicator elements include a chemical indicator element configured to change color if the aspirated fluid from the patient contains protein.

6. The system of claim 1, wherein the port includes a one-way valve configured to enable the fluid from to enter the detection compartment through the port but prevent fluid from exiting the detection compartment through the port.

7. The system of claim 1, further comprising a drain configured to enable fluid to exit the detection compartment.

8. The system of claim 1, further comprising a connector provided on the port that enables the port to be connected to a catheter through which the aspirated fluid can travel to the detection device.

9. The system of claim 1, further comprising a filter compartment that contains a filter configured to filter out air bubbles within liquid.

10. The system of claim 9, wherein the filter is a 2 μm medication filter.

11. The system of claim 9, further comprising an inlet through which medication can be passed into the filter compartment, through the filter, and through the port so as to deliver the medication to a catheter that has been placed within the patient.

12. The system of claim 11, further comprising a one-way valve positioned between the filter compartment and the port that enables fluid to pass exit the filter compartment but prevents fluid from entering the filter compartment.

13. The system of claim 12, wherein both the detection compartment and the filter compartment are in fluid communication with the port.

14. The system of claim 11, further comprising a drain configured to enable fluid to exit the detection compartment and wherein the inlet and the drain are combined to form a single port.

15. A method for confirming placement of a catheter within an epidural space of a patient, the method comprising:
delivering medication through a catheter having a tip that is believed to be located within the epidural space;
aspirating fluid from the patient with the catheter;
delivering the aspirated fluid to a detection compartment of a cerebrospinal fluid detection device that contains one or more indicators configured to indicate if the aspirated fluid contains cerebrospinal fluid; and
confirming that the catheter tip is located within the epidural space if the one or more indicators indicate that the aspirated fluid contains cerebrospinal fluid.

16. The method of claim 15, wherein delivering the aspirated fluid to a detection compartment comprises passing the aspirated fluid through a one-way valve that enables fluid to enter the detection compartment but prevents fluid from exiting the detection compartment.

17. The method of claim 15, wherein the one or more indicators comprise one or more chemical indicator elements configured to indicate when cerebrospinal fluid comes into contact with the chemical indicator elements.

18. The method of claim 17, wherein the one or more chemical indicator elements include a chemical indicator element configured to change color if the aspirated fluid has a pH within a pH range of cerebrospinal fluid.

19. The method of claim 17, wherein the one or more chemical indicator elements include a chemical indicator element configured to change color if the aspirated fluid contains glucose.

20. The method of claim 17, wherein the one or more chemical indicator elements include a chemical indicator element configured to change color if the aspirated fluid contains protein.
